(19) United States
(12) Patent Application Publication
(10) Pub. No.: US 2020/0209162 A1
(43) Pub. Date: Jul. 2, 2020

(54) SMART LABEL ARCHITECTURE WITH ORGANIC LEDS

(71) Applicant: Marco De Angeli, Barzana (IT)

(72) Inventor: Marco De Angeli, Barzana (IT)

(21) Appl. No.: 16/377,311

(22) Filed: Apr. 8, 2019

Related U.S. Application Data
(60) Provisional application No. 62/787,086, filed on Dec. 31, 2018.

(51) Int. Cl. G01N 21/81; G01K 11/12; G01N 33/569

(52) U.S. Cl. CPC G01N 21/81; G01N 2021/7753; G01N 33/56911; G01K 11/125

(57) ABSTRACT

Systems, devices and methods for monitoring a physical property over time are disclosed. A monitoring device in accordance with the present disclosure may comprise an electronic chip assembly, at least one light source, at least one monitoring label comprising a chemical configured to absorb light depending upon at least one physical property, and at least one photodiode configured to collect light emitted by the at least one monitoring label and provide at least one measurement corresponding to the at least one physical property. The at least one measurement may correspond to a past or present status of the at least one physical property. The monitoring device may be communicatively coupled to a user device configured to run an application that collects and store the at least one measurement provided by the at least one photodiode.

### CROSS REFERENCE TO RELATED APPLICATION

This application claims the benefit of U.S. Provisional Application No. 62/787,086 having a filing date of Dec. 31, 2018, which is incorporated by reference as if fully set forth.

### BACKGROUND

Labels for monitoring one or more physical properties, such as temperature, are commonly used in a variety of industries to ensure the safety of food and consumer products. Monitoring labels generally fall within one of two categories: (1) a chemical label or (2) a smart label. Chemical labels typically comprise a substrate and a chemical deposited on the substrate, wherein the chemical changes colors based on one or more physical properties. Chemical labels are inexpensive, but require human interaction. For example, to obtain a measurement of a physical property using a chemical label, a person must remove the monitoring label from a package and provide a visual interpretation of the current color against a color scale.

Smart labels, on the other hand, include electronics such as a sensor, a battery and a memory. The smart label may automatically collect measurement data of a physical property via the sensor at certain time periods, and store the measurement data in a memory. The measurement data stored in the memory may then be retrieved via software. Smart labels provide automatic and accurate measurements, but are expensive. As such, it would be desirable to have an inexpensive label that does not require human intervention.

### SUMMARY

Systems, devices and methods for monitoring a physical property over time are disclosed. A monitoring device in accordance with the present disclosure may comprise an electronic chip assembly, at least one light source, at least one monitoring label comprising a chemical configured to absorb light depending upon at least one physical property, and at least one photodiode configured to collect light emitted by the at least one monitoring label and provide at least one measurement corresponding to the at least one physical property. The at least one measurement may correspond to a past or present status of the at least one physical property. The monitoring device may be communicatively coupled to a user device configured to run an application that collects and store the at least one measurement provided by the at least one photodiode.

### BRIEF DESCRIPTION OF THE DRAWINGS

FIG. 1 is a perspective view of the assembled monitoring device according to an embodiment;

FIG. 2 is an exploded view of the monitoring device according to an embodiment;

FIG. 3 is a diagram of the monitoring device according to an embodiment;

FIG. 4A is perspective view of the monitoring device according to an embodiment;

FIG. 4B is a top view of the monitoring device according to an embodiment;

FIG. 5 is an illustration of a system comprising the monitoring device according to an embodiment;

FIG. 6 is a flowchart for manufacturing the monitoring device according to an embodiment; and

FIG. 7 is a flowchart for monitoring at least one physical property according to an embodiment.

### DETAILED DESCRIPTION OF THE PREFERRED EMBODIMENTS

Examples of different monitoring systems, devices and methods will be described more fully herein with reference to the accompanying drawings. These examples are not mutually exclusive, and features found in one example can be combined with features found in one or more other examples to achieve additional implementations. Accordingly, it will be understood that the examples shown in the accompanying drawings are provided for illustrative purposes only and they are not intended to limit the disclosure in any way. Like numbers refer to like elements throughout.

Described below are systems, devices and methods for monitoring at least one physical property via at least one monitoring label. The systems, devices and methods described may provide inexpensive, automated monitoring of at least one physical property. The systems, devices and methods described herein have a variety of applications, including but not limited to supply chain and logistic monitoring and biomarker detection in medical applications.

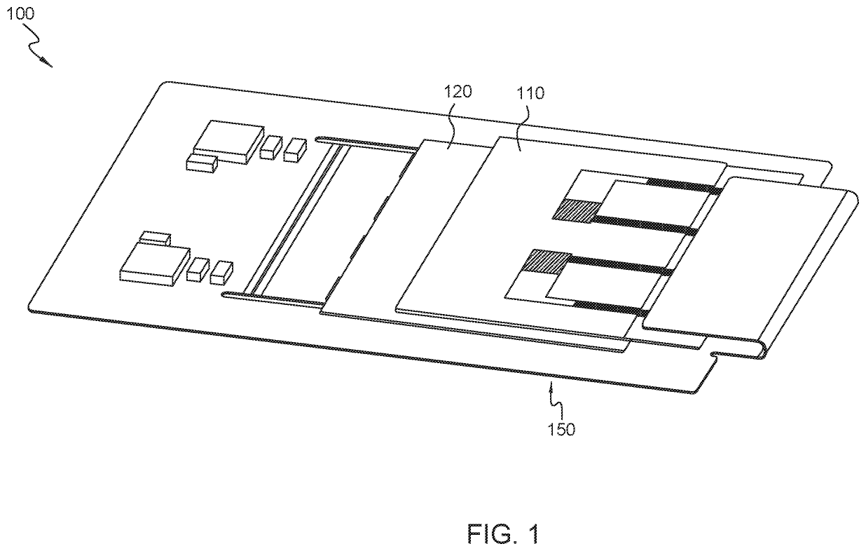

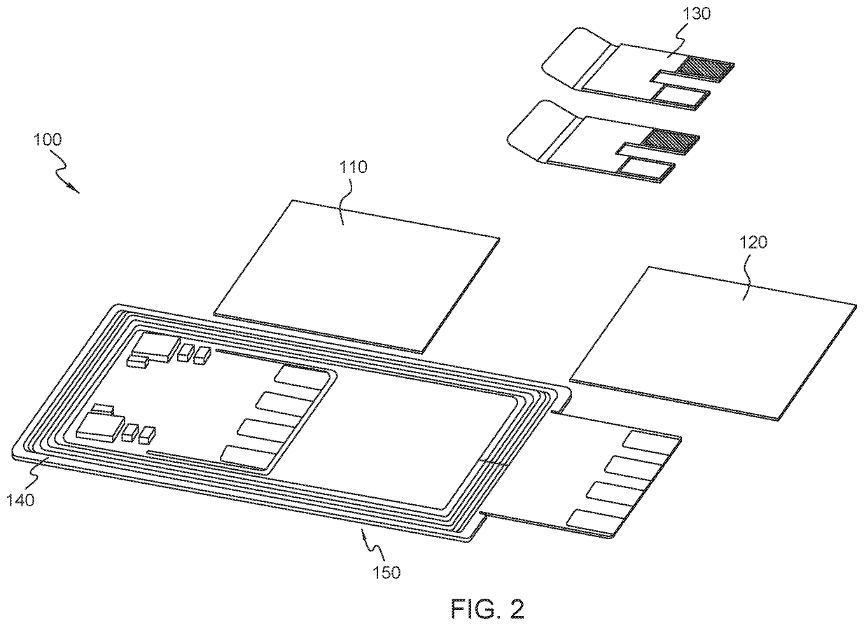

Referring to FIGS. 1 and 2, the monitoring device 100 of the present disclosure may comprise at least one monitoring label 130, at least one light source 120, at least one photodiode 110 and an electric chip assembly 150.

In an embodiment, the at least one monitoring label 130 comprises a substrate and a chemical disposed on the substrate. The chemical disposed on the substrate may be configured to change color based upon at least one physical property. In a further embodiment, the at least one physical property is one or more of temperature, humidity, the presence of one or more bacteria and the presence of one or more molecules. In an embodiment, the chemical may be a thermochromic ink that changes color with exposure to heat. After absorbing a certain amount of heat, the molecular structure of the thermochromic ink may change in such a way that it absorbs and emits light at a different wavelength than at lower temperatures. Therefore, thermochromic ink changes color when temperature increases or decreases.

The chemical disposed on the at least one monitoring label 130 may be reversible or non-reversible. A reversible chemical changes color based on present physical properties. Therefore, a monitoring label comprising a reversible chemical may indicate a current physical property. A monitoring label comprising a reversible chemical may be reusable. A non-reversible chemical, on the other hand, changes color permanently in response to a physical property. Therefore, a monitoring label comprising a non-reversible chemical may indicate a past physical property. For example, a monitoring label comprising a non-reversible thermochromic ink may indicate that a threshold temperature was reached at a certain point. A monitoring label comprising a non-reversible chemical is typically only suitable for one time use.

The at least one monitoring label 130 may be thin and flexible. In an embodiment, the at least one monitoring label is relatively small. For example, the length and height of the at least one monitoring label may be several inches.

The at least one light source 120 may emit light within a wavelength range absorbed by the at least one monitoring label 130. For example, in an embodiment, the at least one light source 120 emits light within a visible wavelength range. Additionally or alternatively, the at least one light source 120 emits light within an invisible wavelength range. The invisible wavelength range encompasses ultraviolet (UV) wavelengths. Additionally or alternatively, the invisible wavelength range encompasses infrared (IR) wavelengths.

The at least one light source 120 may comprise an organic LED (OLED). OLED technology may provide a diffused lighting system that does not require the adoption of complex focusing optics.

The at least one photodiode 110 may be configured to collect light emitted by the at least one monitoring label 130. For example, in an embodiment, the at least one photodiode 110 may overlay the at least one light source 120. In a further embodiment, the at least one monitoring label 130 is located in between the at least one light source 120 and the at least one photodiode 110. As such, light is emitted from the at least one light source 120, absorbed by the at least one monitoring label 130 and then collected by the at least one photodiode 110. Therefore, the amount of light collected by the at least one photodiode 110 may be dependent upon the color of the at least one monitoring label 130. The at least one photodiode 110 converts the collected light into an electrical current to be processed by one or more electrical components of the electric chip assembly 150. As such, the at least one photodiode 110 may provide at least one measurement corresponding to the at least one physical property. In an embodiment, the at least one photodiode 110 may comprise an organic photodiode (OPD).

In an embodiment, the at least one light source 120 may comprise a plurality of light sources 120. In a further embodiment, the plurality of light sources 120 may be arranged in an array. For example, in an embodiment, the at least one light source 120 may be an OLED array. Similarly, the at least one photodiode 110 may comprise a plurality of photodiodes 110. In a further embodiment, the plurality of photodiodes 110 may be arranged in an array. For example, in an embodiment, the at least one photodiode 110 may be an OPD array.

Referring to FIGS. 1 and 2, the at least one light source 120, the at least one monitoring label 130 and the at least one photodiode 110 are assembled an on electronic chip assembly 150. In an embodiment the electronic chip assembly 150 may be a printed circuit board (PCB). In a further embodiment, the electronic chip assembly 150 is a flexible PCB. Alternatively, the electronic chip assembly 150 is a rigid PCB.

In an embodiment, the electronic chip assembly 150 may include a near-field communication (NFC) integrated circuit 140. The NFC integrated circuit 140 may comprise an NFC antenna. An external NFC reader may activate the NFC antenna when the external NFC reader is within the electric field of the NFC integrated circuit 140. The NFC integrated circuit 140 may be configured to wirelessly communicate with the NFC reader. A portable user device, including but not limited to a smartphone, tablet or laptop, may comprise an NFC reader. As such, in an embodiment, the NFC integrated circuit 140 may be configured to wirelessly communicate with a portable user device, as described in more detail below.

The NFC integrated circuit 140 may have energy harvesting capabilities. Energy harvesting is the process by which energy is derived from external sources. The NFC integrated circuit 140 may have passive energy harvesting capabilities. In an embodiment where the NFC integrated circuit 140 has passive energy-harvesting capabilities, energy is scavenged from the NFC reader. Energy harvesting provides a small amount of power for low-energy electronics, such as the at least one light source 120 and the at least one photodiode 110 of the monitoring device 100. In general, energy harvesting is a cheaper power source than a battery. As such, the monitoring device 100 may be lower cost than many smart labels that use a battery as the power source.

In an embodiment, the NFC integrated circuit 140 may have calibration data storage capabilities. Additionally or alternatively, in embodiment, the NFC integrated circuit 140 may have analog to digital conversion capabilities. For example, the NFC integrated circuit 140 may be able to convert the electrical current of the at least one photodiode 110 into a digital signal. The digital signal may then be transmitted wirelessly via NFC to the NFC reader.

In an embodiment, the monitoring device 100 may be configured for biomarker detection in medical applications. For example, in an embodiment, the monitoring device 100 may be configured to monitor the presence of a biomarker via UV fluorescence. For example, the chemical disposed on the at least one monitoring label 130 may be configured to bind with a biomarker. The chemical and the biomarker may absorb light within the UV wavelength range when they are bound together. As such, when the at least one monitoring label 130 is exposed to UV light, the at least one monitoring label 130 may emit a fluorescence when the biomarker is present. In an embodiment, the at least one light source 120 emits UV light and the at least one photodiode 110 is sensitive to light within a visible wavelength range. In an embodiment, the at least one photodiode is sensitive light within a visible green wavelength range. The at least one photodiode 110 may be insensitive to light within the UV wavelength range.

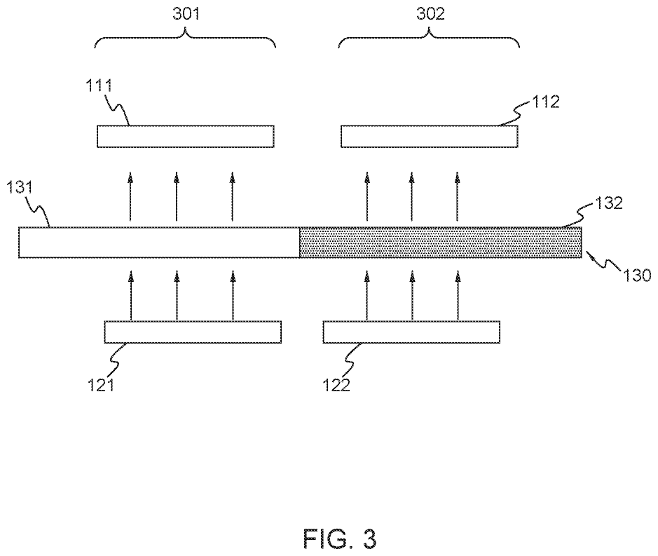

Referring to FIG. 3, in an embodiment, the monitoring device 100 may comprise a reference channel 301. In an embodiment, the system comprises at least two light sources. The at least two light sources may comprise a first light source 121 and a second light source 122. The system may comprise at least two photodiodes. The at least two photodiodes may comprise a first photodiode 111 and a second photodiode 112. The first light source 121 is paired with the first photodiode 111 and the second light source 122 is paired with the second photodiode 112.

In an embodiment, a color changing chemical is not disposed on a reference portion 131 of the at least one monitoring label 130. The reference portion 131 may be disposed between the first light source 121 and the second photodiode 111. The first light source 121, the reference portion 131 and the first photodiode 111 may comprise a reference channel 301. A color changing chemical may be disposed on a sensitive portion 132 of the at least one monitoring label 130. The sensitive portion 132 may be disposed between the second light source 122 and the second photodiode 112. The second light source 122, the sensitive portion 132 and the second photodiode 112 may comprise a sensitive channel 302. The measurements provided via the first photodiode 111 may be used for calibration purposes to improve accuracy of the measurements collected by the second photodiode 112. For example, in an embodiment, the measurements provided via the first photodiode 111 are used for a ratiometric calibration. As such, in this embodiment, the reference channel 301 may be used to compensate for drifts, aging and variability of light emitted from the at least one light source 121.

In an alternate embodiment, a color changing chemical is disposed on both the reference portion 131 and the sensitive portion 132 of the at least one monitoring label. In this embodiment, the device is configured such that the reference portion 131 is not exposed to light emitted by the at least one light source 120. As such, the reference portion 131 may provide a baseline measurement that may be used to compensate for process driven variability. The baseline measurement of the reference portion 131 may compensate for factors that impact the absorbance of the color changing chemical disposed on the reference portion 131 and the sensitive portion 132 of the at least one monitoring label 130. For example, the reference portion 131 may provide information regarding the thickness of the chemical and the concentration of the chemical.

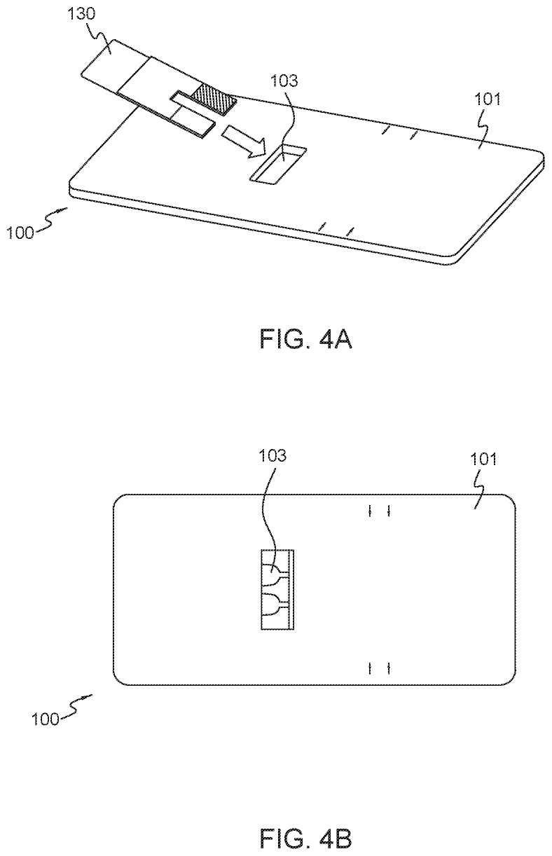

Referring to FIGS. 4A and 4B, the at least one light source 120, the at least one photodiode 110 and the electric chip assembly 150 may be enclosed in a protective case 101. In an embodiment, the protective case 101 may be comprised of a plastic material. In an embodiment, the protective case 101 may comprise at least one opening 103. The at least one opening 103 may be configured to receive the at least one monitoring label 130.

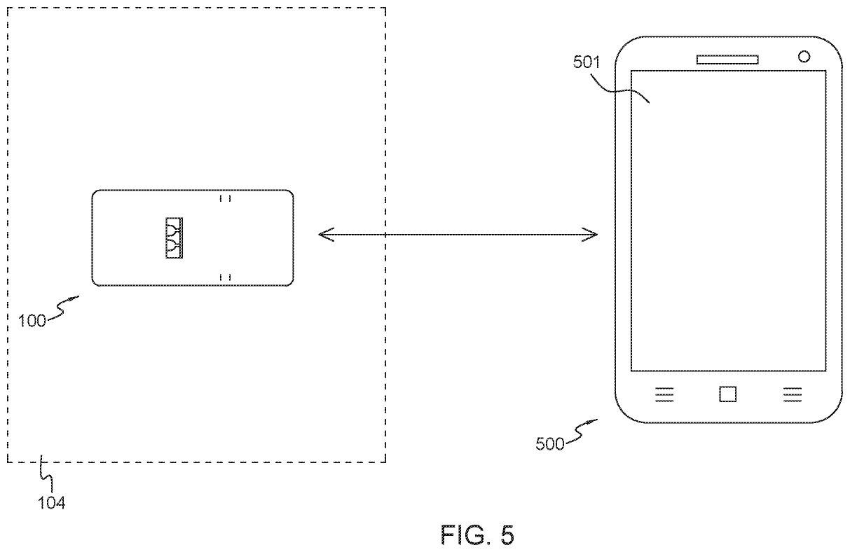

With reference to FIG. 5, in an embodiment, the monitoring device 100 may be affixed to a product to be monitored 104. Further, the monitoring device 100 may be communicatively coupled to a user device 500. In an embodiment, the user device 500 may be a mobile device, such as a smart phone, tablet, portable computer, personal desktop computer or the like. In an embodiment, the monitoring device 100 may be communicatively coupled to the user device 500 wirelessly. For example, the monitoring device 100 may be communicatively coupled to the user device 500 through NFC, as discussed above. However, the monitoring device 100 may be communicatively coupled to the user device 500 through other wireless communication protocols such as radio-frequency identification (RFID), Bluetooth, Wi-Fi, Bluetooth Low Energy (BLE) or Long-Term Evolution (LTE). These wireless communication protocols are known in the art and are not discussed in detail here. As will be appreciated by a person having ordinary skill in the art, this list is meant to be illustrative and not exhaustive, and the monitoring device may be communicatively coupled to the user device 500 through other wireless communication protocols.

In an embodiment, the monitoring device 100 may be configured to transmit the at least one measurement provided by the photodiode 110 to the user device 500. The user device 500 may be configured to receive the at least one measurement provided by the at least one photodiode 100. The at least one measurement may be transmitted to the user device 500 automatically. For example, the at least one measurement may be transmitted to the user device 500 automatically when the user device 500 is within the electric field of the NFC integrated circuit 140. Additionally or alternatively, at least one measurement may be transmitted to the user device 500 on demand when commanded by an input.

In an embodiment, the user device 500 may be configured to run an application that collects the at least one measurement of the at least one photodiode 110. In a further embodiment, the user device 500 may have a graphical user interface 501 for displaying the at least one measurement provided by the at least one photodiode 110.

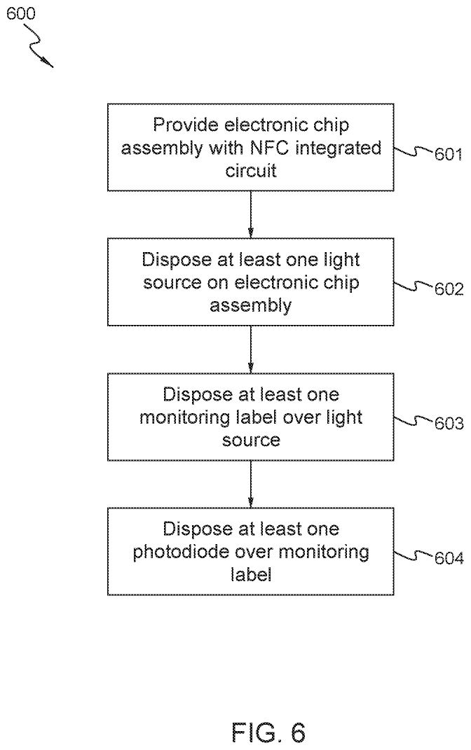

Referring to FIG. 6, a method 600 for manufacturing the system 100 of the present disclosure is shown. At step 601, an electronic chip assembly 150 is provided. In an embodiment, the electronic chip assembly 150 may be a printed circuit board. In an embodiment, the electronic chip assembly 150 may include a NFC integrated circuit 140. At step 602, at least one light source 120 is disposed on the electronic chip assembly 150. At step 603, the at least one monitoring label 130 is disposed over the at least one light source 120. A chemical may be disposed on the at least one monitoring label 130 that changes color based on at least one physical property. In a further embodiment, the at least one physical property is one or more of temperature, humidity, the presence of one or more bacteria and the presence of one or more molecules. Additionally or alternatively, the chemical may be configured to bind with a biomarker. At step 604, at least one photodiode 110 is disposed over the at least one monitoring label 130. In an embodiment, the method 600 may further comprise enclosing the at least one light source 120, the at least one photodiode 110 and the electric chip assembly 150 in a protective case 103.

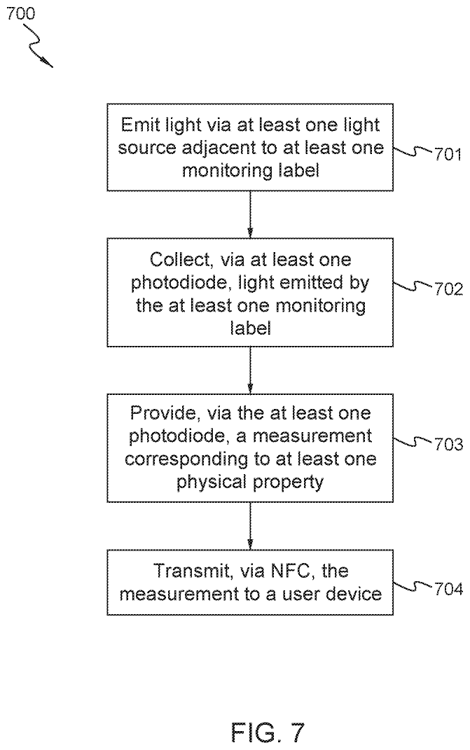

Referring to FIG. 7, a method 700 for monitoring at least one physical property is shown. This method 700 for monitoring may be used in conjunction with the monitoring device 100 of the present disclosure. At step 701, light is emitted from at least one light source 120 that is adjacent to at least one monitoring label 130. The light emitted may be within a wavelength range absorbed by the at least one monitoring label 130. At step 702, the at least one photodiode 110 collects light emitted by the at least one monitoring label 130. A chemical may be disposed on the at least one monitoring label 130 that changes color based on at least one physical property. In an embodiment, the at least one physical property is one or more of temperature, humidity, the presence of one or more bacteria and the presence of one or more molecules. Additionally or alternatively, the chemical may be configured to bind with a biomarker. At step 703, the at least one photodiode 110 provides at least one measurement corresponding to the at least one physical property. At step 704, the at least one measurement provided by the at least one photodiode 110 is transmitted to the user device 500. The at least one measurement provided by the photodiode 110 may be transmitted to the user device 500 automatically. For example, the at least one measurement may be transmitted to the user device 500 automatically when the user device 500 is within the electric field of the NFC integrated circuit 140. Additionally or alternatively, at least one measurement may be transmitted to the user device 500 on demand when commanded by an input.

In an embodiment, the electronic chip assembly 150 may include an NFC integrated circuit 140. As such, the method 700 may further comprise activating, via the NFC integrated circuit, the at least one light source 120 and the at least one photodiode 110 through energy harvesting. Additionally or alternatively, the method 700 may further comprise converting, via the NFC integrated circuit 140, an electrical current produced by the at least one photodiode 110 into a digital signal. Additionally or alternatively, the method may further comprise storing, via the NFC integrated circuit 140, calibration data.

Although features and elements are described above in particular combinations, one of ordinary skill in the art will appreciate that each feature or element can be used alone or in any combination with the other features and elements.

What is claimed is:

1. A monitoring device comprising:
an electronic chip assembly;
at least one light source;
at least one monitoring label comprising a chemical configured to absorb light depending upon at least one physical property; and
at least one photodiode configured to collect light emitted by the at least one monitoring label and provide at least one measurement corresponding to the at least one physical property.

2. The monitoring device of claim 1, wherein the at least one monitoring label is disposed between the at least one light source and the at least one photodiode.

3. The monitoring device of claim 1, wherein the at least one light source emits light within a visible wavelength range.

4. The monitoring device of claim 1, wherein the at least one light source emits light within an invisible wavelength range.

5. The monitoring device of claim 4, wherein the at least one light source emits light within an ultraviolet (UV) wavelength range.

6. The monitoring device of claim 5, wherein:
the at least one physical property is a biomarker binding with the chemical; and
the at least one photodiode is sensitive to light within a visible wavelength range.

7. The monitoring device of claim 6, wherein the at least one photodiode is sensitive to a visible green wavelength range and insensitive to light within the UV wavelength range.

8. The monitoring device of claim 4, wherein the at least one light source emits light within an infrared (IR) wavelength range.

9. The monitoring device of claim 1, wherein the at least one light source is an organic LED and the at least one photodiode is an organic photodiode.

10. The monitoring device of claim 1, wherein the at least one light source comprises a plurality of light sources and the at least one photodiode comprises a plurality of photodiodes.

11. The monitoring device of claim 10, wherein the plurality of photodiodes comprises at least a first photodiode and a second photodiode, the first photodiode configured to collect light emitted by a portion of the at least one monitoring label on which the chemical is not disposed and the second photodiode is configured to collect light emitted by a portion of the at least one monitoring label on which the chemical is disposed.

12. The monitoring device of claim 11, wherein the measurement provided by the first photodiode is used as a reference measurement in a ratiometric calibration.

13. The monitoring device of claim 1, wherein the device is configured such that a portion of the at least one monitoring label on which the chemical is disposed is not exposed to the light emitted by the at least one light source, and the portion of the at least one monitoring label is used as a reference measurement.

14. The monitoring device of claim 1, wherein the electronic chip assembly comprises a near-field communication (NFC) integrated circuit with one or more of energy harvesting, analog to digital conversion and calibration data storage cap abilities.

15. The monitoring device of claim 1, wherein the at least one physical property is one or more of temperature, humidity, the presence of one or more bacteria or the presence of one or more molecules.

16. A system comprising:
a monitoring device comprising:
an electronic chip assembly;
at least one light source;
at least one monitoring label comprising a chemical configured to absorb light depending upon at least one physical condition; and
at least one photodiode configured to collect light emitted by the at least one monitoring label and provide at least one measurement corresponding to the at least one physical property; and
a user device communicatively coupled to the monitoring device.

17. The system of claim 16, wherein the user device is communicatively coupled to the monitoring device via NFC.

18. The system of claim 17, wherein the user device comprises an NFC reader and the electronic chip assembly of the monitoring device comprises an NFC antenna.

19. A method for monitoring at least one physical property comprising:
emitting light via at least one light source adjacent to at least one monitoring label;
absorbing, via a chemical disposed on the at least one monitoring label, the light emitted by the at least one light source;
collecting, via at least one photodiode, light emitted by the at least one monitoring label;
providing, via the at least one photodiode, at least one measurement;
transmitting, via NFC, the at least one measurement collected by the at least one photodiode to a user device.

20. The method of claim 19, wherein transmitting occurs automatically when an NFC reader is within the electrical field of an NFC antenna or on demand when commanded by an input.
